(19) United States
(12) Patent Application Publication
(10) Pub. No.: US 2010/0069872 A1
(43) Pub. Date: Mar. 18, 2010

(54) ELASTIC ABSORBENT ARTICLE AND A METHOD FOR ITS MANUFACTURE

(76) Inventor: Åsa Lindström, Goteborg (SE)

Correspondence Address: BUCHANAN, INGERSOLL & ROONEY PC, POST OFFICE BOX 1404, ALEXANDRIA, VA, 22313-1404, US

(21) Appl. No.: 12/531,331

(22) PCT Filed: Mar. 16, 2007
(86) PCT No.: PCT/SE07/00263

§ 371 Date: Sep. 15, 2009

(51) Int. Cl. A61F 13/45

(52) U.S. Cl. 604/385.22; 604/385.23; 156/160

(57) ABSTRACT

An absorbent article includes an elastic web material having an aperture formed therein, and an absorbent unit attached to a wearer facing side of the elastic web material so as to cover the aperture. The elastic web material surrounds the absorbent unit on all sides thereof. The absorbent unit is attached to the elastic web material with the elastic web material being in an extended state of at least 30% in both the longitudinal and transverse directions of the article.

### TECHNICAL FIELD

The present invention refers to a disposable absorbent article comprising an elastic web material. The article may be an incontinence pad, a sanitary napkin, a diaper, a pant-like article such as a pant diaper, incontinence pant or sanitary pant. The invention further refers to a method for manufacturing the article.

### BACKGROUND OF THE INVENTION

Disposable absorbent articles exist in many different configurations such as incontinence pads and sanitary napkins intended to be worn in an undergarment and often are provided with adhesive strings intended to be fastened to the undergarment, diapers provided with fastening means adapted to fasten the diaper around the waist of the wearer and pant-type articles such as pant diapers, incontinence pants and sanitary pants capable of being pulled up and down over the hips of the wearer. It is known to make parts of or the entire absorbent article elastic in order to improve fit, comfort and security against leakage. Elasticity can be imparted by elastic members, such as elastic threads, contractably affixed between the inner and outer coversheet of the article or by making parts of the article of an elastic laminate material.

U.S. Pat. No. 6,552,245 discloses an extensible outer cover for an absorbent article which provides a certain permanent deformation when subjected to a tensile force. The extensible outer cover comprises a necked laminate in the form of one layer of a necked non-elastic film and one layer of an elastic film. The films may be breathable.

WO 03/047488 discloses an elastic laminate comprising an elastic film which on opposite sides is bonded to first and second non-elastic fibrous layers. The laminate is made by bonding the non-elastic fibrous layers to the elastic film layer and subsequently stretching the composite material, causing the non-elastic materials to break. The elastic film material may be of a breathable material. The laminate may be incorporated in an absorbent article.

Further examples of absorbent articles which in part are made of elastic laminates are found in U.S. Pat. No. 6,476,289 and JP 10043235.

WO2005/122985 and WO2006/093439 refer to pant-like absorbent articles comprising an absorbent unit in the crotch region and a chassis in the form of a front and a back panel. The chassis comprises an outer coversheet in the form of an elastic laminate. The absorbent unit, which bridges the front and back panels, comprises an absorbent core, a liquid impermeable backsheet on the garment-facing side of the absorbent core and a liquid permeable topsheet on the wearer-facing side of the absorbent core.

US 2005/0020992 refers to a disposable undergarment comprising an elastic web material having a cut-out therein to form a front and a back panel and having an absorbent insert attached to the elastic web material covering the cut-out and bridging the front and back panels.

### SUMMARY OF THE INVENTION

The object of the present invention is to provide an absorbent article comprising an elastic web material and an absorbent unit attached thereto and wherein the elastic properties of the elastic web material are maintained and the article has a neat fit and appearance. The article comprises an elastic web material having an aperture formed therein, and an absorbent unit attached to a wearer facing side of the elastic web material so as to cover said aperture. The elastic web material surrounds the absorbent unit on all sides thereof. The absorbent unit is attached to the elastic web material with the elastic web material being in an extended state of at least 30% in both the longitudinal and transverse direction of the article.

In one aspect of the invention the absorbent unit comprises an absorbent core and the aperture in the elastic web material, when this is in said extended state of at least 30% when being attached to the absorbent unit, is smaller then but not more than 50% smaller than the surface area of the absorbent unit. In a further aspect the aperture, when the elastic web material is in said extended state when being attached to the absorbent unit, is also smaller than the absorbent core.

According to one embodiment the absorbent core has a shape similar to the shape of said aperture when the elastic web material is in said extended state of at least 30%, so that the contours of the aperture substantially follow the contours of the absorbent core.

In one aspect of the invention the absorbent unit is attached to the elastic web material with the elastic web material being in an extended state of at least 40%, preferably at least 50%, in at least one of the longitudinal, y, or transverse, x, direction of the article. In a further aspect the elastic web material has a higher extension in longitudinal direction, y, than in transverse direction, x, when being attached to the absorbent unit.

According to a further embodiment the absorbent unit comprises an inner coversheet and an outer coversheet enclosing said absorbent core. In a still further embodiment the outer coversheet has an external garment facing side comprising a fibrous web. It will in this case be more discrete and not differ so much from the surrounding elastic web material if this also has a fibrous web as external surface.

In one aspect of the invention the article is a pant type absorbent article such as a pant diaper, a sanitary pant or incontinence pant said article comprising said absorbent unit, and a chassis surrounding the absorbent unit, said chassis comprising front, back and waist regions, while the absorbent unit is located at least in the crotch region of the article, said article in at least a part of the chassis comprises an outer coversheet in the form of said elastic web material having said aperture formed therein coinciding with the absorbent unit.

In a further aspect the elastic web material extends beyond the peripheral edges of the absorbent unit around all sides thereof including in the crotch region of the article. The elastic web material may in this case serve as a sealing in the crotch area, making additional leg and/or crotch elastics redundant.

The invention further refers to a method for manufacturing an absorbent article comprising:
providing an elastic web material and forming an aperture therein;
stretching the elastic web material at least 30% in both a longitudinal and a transverse direction;
providing an absorbent unit and attaching said absorbent unit to the elastic web material while this is in its stretched state and so that the absorbent unit covers the aperture in the elastic web material.

It is understood that the aperture may be formed in the elastic web while this is either in a relaxed or stretched condition, thus the two first mentioned method steps above may be reversed, so that the web is first stretched and then the aperture is formed therein. It may also be feasible to have the elastic web material stretched to a lower extent when forming the aperture therein and then stretching it further before attaching the absorbent unit thereto.

### DEFINITIONS

### Absorbent Article

The term “absorbent article” refers to products that are placed against the skin of the wearer to absorb and contain body exudates, like urine, faeces and menstrual fluid. The invention mainly refers to disposable absorbent articles, which means articles that are not intended to be laundered or otherwise restored or reused as an absorbent article after use.

### Liquid Previous Inner Cover

The inner liquid permeable cover can consist of a nonwoven material, e g spunbond, meltblown, carded, hydroentangled, wetlaid etc. Suitable nonwoven materials can be composed of natural fibers, such as woodpulp or cotton fibres, manmade fibres, such as polyester, polyethylene, polypropylene, viscose etc. or from a mixture of natural and manmade fibres. The inner cover may further be composed of tow fibres, which may be bonded to each other in a bonding pattern, as e.g. disclosed in EP-A-1 035 818. Further examples of materials suitable for inner covers are porous foams, apertured plastic films etc. The materials suited as inner cover materials should be soft and non-irritating to the skin and intended to be readily penetrated by body fluid, e.g. urine or menstrual fluid. The inner cover may be different in different parts of the absorbent article.

### Liquid Impervious Outer Cover

The liquid impervious outer cover is of a liquid impervious material, such as a thin plastic film, e.g. a polyethylene or polypropylene film, a nonwoven material coated with a liquid impervious material, a hydrophobic nonwoven material, which resists liquid penetration or a laminate comprising plastic films and nonwoven materials. The outer cover material may be breathable so as to allow vapour to escape from the absorbent core, while still preventing liquids from passing therethrough. Examples of breathable outer cover materials are porous polymeric films, nonwoven laminates from spunbond and meltblown layers, laminates from porous polymeric films and nonwovens.

### Absorbent Core

The absorbent core can be of any conventional kind. Examples of commonly occurring absorbent materials are cellulosic fluff pulp, tissue layers, highly absorbent polymers (so called superabsorbents), absorbent foam materials, absorbent nonwoven materials or the like. It is common to combine cellulosic fluff pulp with superabsorbents in an absorbent body. It is also common to have absorbent bodies comprising layers of different material with different properties with respect to liquid receiving capacity, liquid distribution capacity and storage capacity. The thin absorbent bodies, which are common in for example baby diapers and incontinence guards, often comprise a compressed mixed or layered structure of cellulosic fluff pulp and superabsorbent. The size and absorbent capacity of the absorbent core may be varied to be suited for different uses such as for infants or for incontinent adults.

### Extensibility and Elastic Extensibility

Reference to the percentage of extensibility is indicative of the amount of extensibility over and above the contracted length of the material. Thus, by way of example, if a strip of material having a contracted length of 10 cm is extensible by 50%, it will reach an extended length of 15 cm. Furthermore, when used herein, the expression “elastic extensibility” implies that the material will undergo a plastic deformation of no more than 20%. Thus, for the example in which the 10 cm long strip of material is extended by 50% to 15 cm, it will revert to a length of no more than 12 cm when the force causing the extension is removed.

### BRIEF DESCRIPTION OF THE DRAWINGS

The invention will in the following be closer described with reference to some embodiments shown in the accompanying drawings.

FIG. 1 is a perspective view of a pant diaper.

FIG. 2 is a plan view of the pant diaper in its flat, uncontracted state prior to formation as seen from the garment facing side.

FIG. 3 is a cross section according to the line III-III in FIG. 2.

FIG. 4 is a cross section through an elastic laminate according to the invention.

FIG. 5 is a plan view of an absorbent article in the form of an incontinence pad or sanitary napkin in its flat, uncontracted state.

FIG. 6 is a cross section according to the line VI-VI in FIG. 5.

FIG. 7 is a schematic plan view illustrating part of a method for manufacturing the pant diaper.

FIG. 8 is a schematic plan view illustrating part of an alternative method for manufacturing an absorbent article.

### DETAILED DESCRIPTION OF PREFERRED EMBODIMENTS

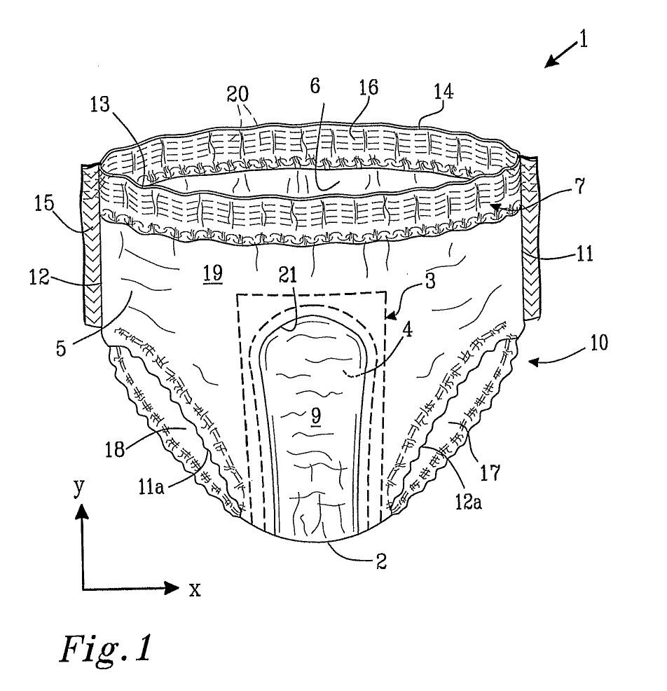

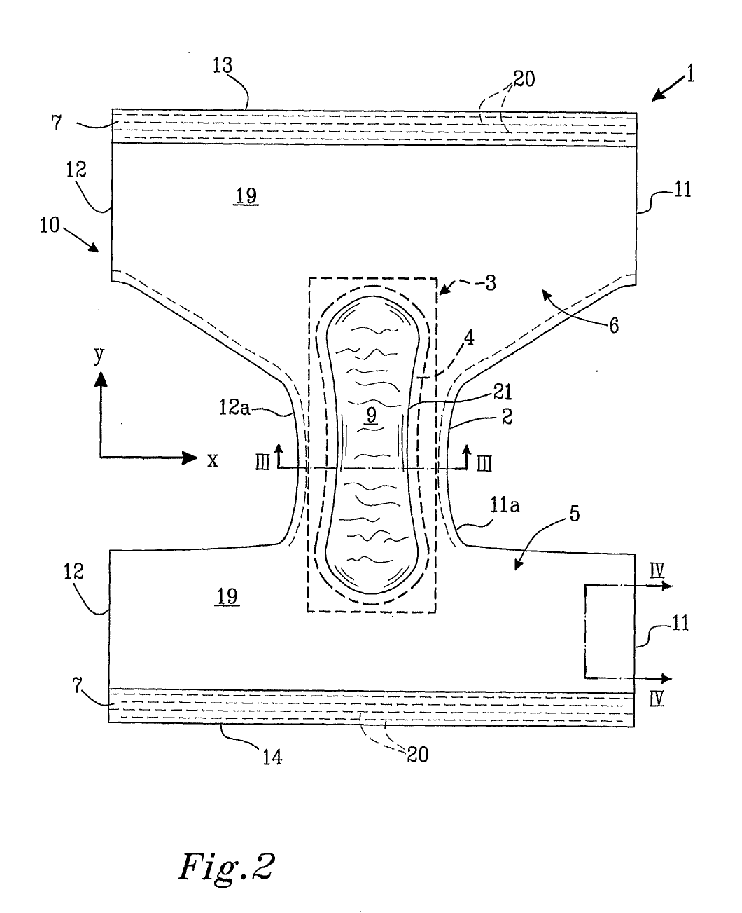

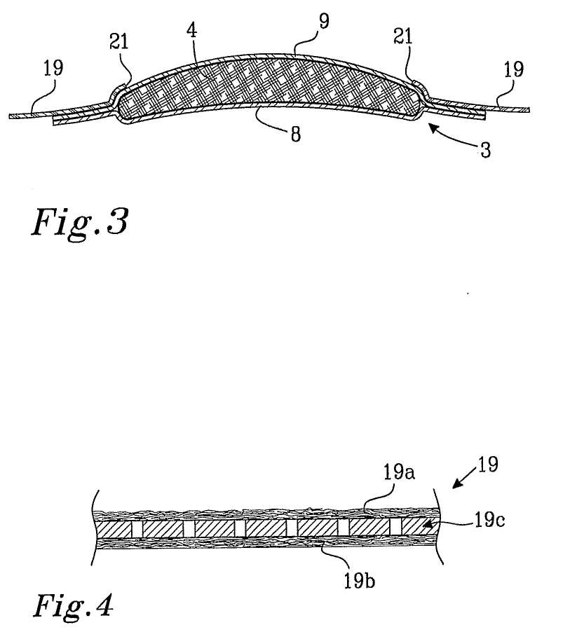

The absorbent pant-like article 1 disclosed in FIG. 1-3 is intended to enclose the lower part of the wearer's trunk like a pair of absorbent pants and can be worn by an infant or an incontinent adult. It comprises a crotch region 2, having an absorbent unit 3 therein, said absorbent unit comprising an absorbent core 4, a liquid pervious inner cover 8 and a liquid impervious outer cover 9, and a chassis 10 outside the absorbent unit 3. The chassis 10 comprises those parts of the front 5, back 6 and waist regions 7 of the pant that are located outside the absorbent unit 3.

The article is defined by a pair of longitudinal side edges 11 and 12 having an inwardly directed leg contour 11a, 12a, and a pair of transverse waist edges 13 and 14. The front 5 and back regions 6 are joined to each other along their longitudinal edges by ultrasonic welds, glue strings or the like forming side seams 15 to define a waist-opening 16 and a pair of leg-openings 17 and 18. The absorbent unit 3 is located in the narrow crotch region 2 of the article and extends into the front 5 and back regions 6. The article has a longitudinal direction, y, and a transverse direction, x.

The entire or at least a part of the chassis region comprises a coversheet 19 of a soft and comfortable material in the form of an elastic web material. The elastic web material for the purpose of the present invention should have an elastic extensibility, as defined above, of at least 30% beyond its original dimension both in a longitudinal and a transverse direction when subjected to a tensile force. It should have a permanent deformation of not more than 20% after the force causing the extension is removed, which means that if it has been extended from 10 to 13 cm, it should have a permanent deformation of not more than 20% of 3 cm, i.e. not more than 0.6 cm.

It may be preferred that the elastic web material has an elastic extensibility of at least 40% and preferably at least 50% in longitudinal and/or transverse direction of the article. It may have different elastic extensibility in longitudinal and in transverse direction.

The elastic web material may for example be in the form of an elastic laminate composed of first and second outer layers of fibrous material 19a and 19b and a middle elastic film layer 19c located between said fibrous layers, as shown in FIG. 4. Examples of suitable fibrous materials are carded webs and spunbond materials. In one embodiment the basis weight of the fibrous material layers is between 10 and 35 g/m², preferably between 12 and 30 g/m², more preferably between 15 and 25 g/m². Examples of suitable polymers used in the fibrous materials are polyethylene, polyesters, polypropylene and other polyolefin homopolymers and copolymers. Natural fibres, for example cotton, may also be used. A mixture of polymers can contribute to a higher flexibility of the nonwoven layer, and through this, give the nonwoven material a higher elongation at maximum load. A mixture of polyethylene and polypropylene polymers has proved to provide good results in this respect. A mixture of fibers of different polymers is also possible.

The middle layer 19c is according to one embodiment of the invention an apertured elastic film having a basis weight between 20 and 80 g/m², preferably between 20 and 60 g/m². The film may be of any suitable elastic polymer, natural or synthetic. Some examples of suitable materials for the elastic film are low crystallinity polyethylenes, metallocene-catalyzed low crystallinity polyethylene, ethylene vinyl acetate copolymers (EVA), polyurethane, polyisoprene, butadiene-styrene copolymers, styrene block copolymers, such as styrene/isoprene/styrene (SIS), styrene/butadiene/styrene (SBS), or styrene/ethylene-butadiene/styrene block copolymer. Blends of these polymers may also be used as well as other modifying elastomeric or non-elastomeric materials. One example of a suitable film is an apertured three-layer elastomeric film of PE-SEBS-PE.

For i.a. reasons of comfort, it is advantageous if the total basis weight of the laminate can be kept low. Thus, although a total basis weight of about 150 g/m² is acceptable, a total basis weight of 100 g/m² or less, for example no more than 90 g/m², is preferred.

The elastic laminate 19 may be manufactured according to the method disclosed in WO 03/047488, wherein one spunbond layer is applied to the film in a tacky state and will thus bond to the film layer, while the other spunbond layer is adhesively laminated to the film layer, using for example a pressure sensitive hot melt adhesive. Alternatively the laminate is manufactured according to a modified version of this known method, wherein the modification involves that the laminate is incrementally stretched (through intermeshing gears, IMG), to a point below the elongation at peak load of at least one of the non-elastic nonwoven layers to retain some strength for at least one of the nonwoven layers. The other layer may also be stretched to a point below its elongation at peak load, or to a point at which it will tear during stretching.

The method disclosed in WO 03/047488 involves stretching of the laminate above the point of failure of the fibrous material, so that the non-elastic layers break completely. Therefore, as described in WO 03/047488, the elongation of the laminate is not limited by the stretch modulus of the non-elastic material.

Elasticity in both the longitudinal direction and the transverse direction may be imparted to the laminate in the manner disclosed in, for example, WO-A-95/04654.

To provide additional wearer comfort, the elastic laminate 19 may be breathable and have a Water Vapour Transmission Rate according to ASTM E96-00 of at least 1500 g/m²-24 h, preferably at least 3000 g/m²-24 h.

Further examples of elastic laminates are stretch-bonded laminates in which a nonelastic material is joined to an elastic material while the elastic material is in a stretched condition, so that when the elastic material is relaxed, the nonelastic material gathers between the locations where it is bonded to the elastic material. It is also known to laminate (or bond) a necked material to an elastic sheet to produce a neck bonded laminate which is capable of stretch and recovery in the cross-machine direction. This process involves an elastic member being bonded to a non-elastic member while only the non-elastic member is extended in one direction (usually the machine direction) and necked in the transverse direction, so as to reduce its dimension in the direction orthogonal to the extension. It is further known to utilize intermeshing grooved rolls or discs for stretching nonwoven webs. For instance, it is known to use grooved rolls generally to stretch a laminate formed from an elastic and a non-elastic (extendable) web, for example a nonwoven, in order to form an elastic laminate.

Additional examples of bi-directionally elastically extensible material which may be adapted for use in the present invention is described in the art. Examples include that which is disclosed in US-A1-2003/0105446, WO-A-95/29810, EP-A-0 432 763 and WO-A-95/32093.

However it is understood that other types of elastic web materials may be used, such as elastic nonwoven materials, nonwoven materials which per se are inelastic, but which have been elastified by means of elastic threads etc. The elastic web materials may comprise one layer or two or more layers that have been laminated.

An elastic waist band is further provided in the waist region 7, said waist band in one embodiment comprises a substantially non-elastic nonwoven material that is elasticized by elongate elastic members 20 such as elastic threads, contractably affixed between material layers, such as nonwoven materials.

The elastic web material 19 has an aperture 21 in the crotch region 2. The absorbent unit 3 comprising the absorbent core 4, inner cover 8 and outer cover 9, is attached to the wearer facing side of the elastic web material 19 so as to cover said aperture 21. The absorbent unit 3 has a larger surface area than the aperture 21 when the elastic web material is in the stretched condition used during attachment of the absorbent unit and the elastic web material 19 is attached to the absorbent unit 3 around all sides thereof. Preferably the elastic web material 19 alone, without having part of the absorbent unit 3 attached thereto, surrounds the absorbent unit 3 around all sides, including along the leg contour edges 11a, 12a, in the crotch region 2. The elastic web material 19 may herewith form an elastic sealing serving as leg and/or crotch elastics in said area making additional leg and/or crotch elastic threads redundant.

Thus the elastic web material 19 should cover at least those parts of the chassis having the aperture 21 formed therein, usually the crotch region and optionally also parts of the front and back regions of the article. Other areas of the chassis may also be formed from the elastic web material, or from another material.

The absorbent unit 3 is attached to the elastic web material 19 with the elastic web material being in an extended state of at least 30% both in longitudinal, y, and transverse, x, direction of the article. This involves that upon relaxation of the elastic web material 19 this will substantially maintain its elastic properties also in the areas immediately outside the absorbent unit 3, which normally is inelastic. The elastic web material in the chassis will, when the stretch force is removed, contract around the absorbent core 4, so that the chassis becomes somewhat bowl-shaped around the absorbent core. In an alternative embodiment the absorbent unit 3 may also contain elastic material.

It may for some articles be desired that the elastic web material 19 is extended at least 40%, preferably at least 50% in longitudinal, y, and/or transverse, x, direction of the article when attaching the absorbent unit 3 to the elastic web material. The elastic web material 19 may have different degrees of extension in longitudinal and transverse direction when attaching the absorbent unit 3 thereto. It may for example have a higher degree of extension in the longitudinal than in the transverse direction.

When the elastic web material 19 should serve as leg and/or crotch elastics around the leg openings of the pant article it may be desired that the elastic web material 19 is extended at least 50% in its longitudinal direction, y, when attaching the absorbent unit 3 thereto.

The aperture 21 when the elastic web material 19 is in its extended state during attachment of the absorbent unit 3 should have a size that is smaller than the absorbent unit 3 and preferably smaller than the absorbent core 4 as well. It is preferred that the aperture 21 in said extended state has a size, as measured as its surface area, which is between 50 and 95%, preferably between 60 and 90% of the surface area of the absorbent unit 3. It is further preferred that the absorbent core 4 has a shape similar to the shape of the aperture 21 when the elastic web material 19 is in said extended state, so that the contours of the aperture substantially will follow the contours of the absorbent core.

The absorbent unit 3 is attached to the elastic web material 19 by any suitable method, such as ultrasonic welding, laser welding, heat bonding, gluing, stitch-bonding etc. The part of the absorbent unit 3 that is exposed through the aperture 21 as seen from the garment facing side of the article will protrude somewhat there through.

In order to make the visible part of the absorbent unit 3 as discrete as possible it is desired that at least those parts of the outer cover 9 that are visible through the aperture has an appearance that resembles the garment facing side of the elastic web material 19. Thus in case the elastic web material 19 has a fibrous layer as garment facing side, the outer cover, or at least those parts thereof that are visible through the aperture 21, should have a fibrous layer as outer garment facing layer. The fibrous layers on the garment facing side of the elastic web material 19 and on the outer cover 9 that is visible through the aperture 21 preferably have a similar appearance and feel, so that the difference between them is hardly noticable.

In one embodiment the surface area of the absorbent core 4 amounts to no more than 30%, preferably not more than 20%, of the total surface area of the article, as measured in a flat, extended, state of the article. The elastic web material 19 constitutes the sole component of at least parts of the front and back regions 5 and 6 and preferably also along the leg contour edges 11a and 12a in the crotch region 2, so as to form leg and/or crotch elastics, as described above.

No additional elasticized side panels joining the front and back regions 5 and 6 are needed when using the elastic web material 19.

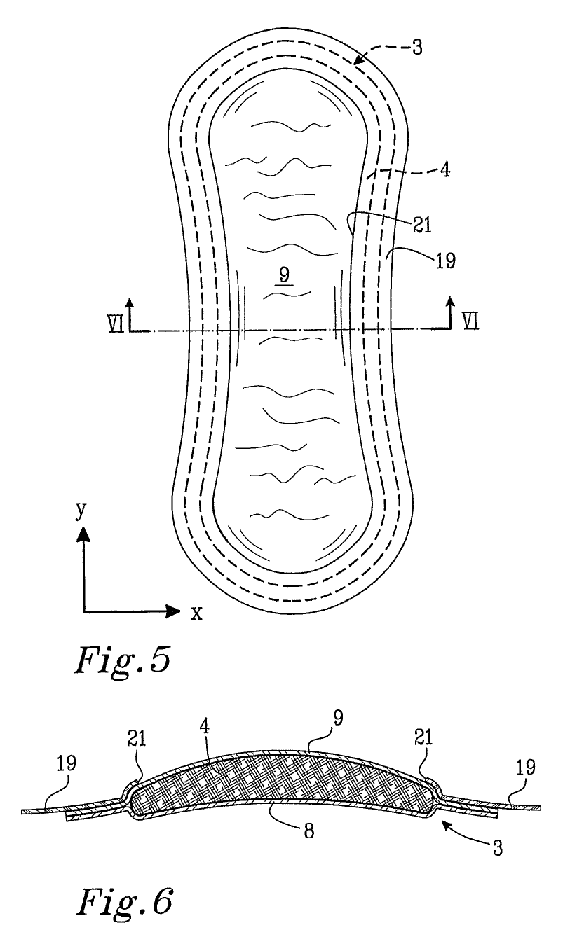

FIGS. 5 and 6 show another type of absorbent article in the form of an incontinence pad or sanitary napkin intended to be used as an insert or a pad in a pair of pants or underwear. FIG. 5 is a plan view of the article in its flat, uncontracted state as seen from the garment facing side. FIG. 6 is a cross section according to the line VI-VI in FIG. 5.

The article comprises an absorbent unit 3 having an absorbent core 4, an inner liquid pervious cover 8 and an outer liquid impervious cover 9. The article further comprises an elastic web material 19 of the same kind as disclosed above. The inner cover 8 and the outer cover 9 extend outwardly beyond the peripheral edges of the absorbent core 4 and have their inner surfaces bonded to each other, e.g. by gluing or welding by heat or ultrasonic. The inner and outer cover materials may further be bonded, e.g. by adhesive, to the absorbent core 4.

The elastic web material 19 extends around the periphery of the article and extends outside the absorbent unit 3. The elastic web material 19 has an aperture 21 formed therein and the absorbent unit 3 is attached to the wearer facing side of the elastic web material 19 so as to cover said aperture 21. The attachment of the absorbent unit 3 to the elastic web material 19 is, in the same manner as described above, attached to the elastic web material while this is an extended state in both longitudinal, y, and transverse, x, direction of the article. The aperture 21 has in said extended state a size which is smaller than, preferably has a surface area which is between 50 and 95%, more preferably between 60 and 90% of the surface area of the absorbent unit 3.

The elastic web material 19 extending around the absorbent unit 3 will when the extension force is removed tend to contract and cause the entire article to form a bowl-shape, which will improve body contact and leakage security. Therefore leg and/or crotch elastics in the form of elastic threads may not be needed.

The article may in a known manner be provided with fastening means in the form of adhesive strings on the outside of the outer cover to be fastened to the pants or underwear.

It is however understood that the absorbent article according to the invention may be of any other known type, such as a diaper or an incontinence garment, intended to be fastened together around the waist of the wearer by fastening means, such as adhesive tape or hook-and-loop fasteners, to assume a pant-like shape. The article may also be a belted article having belt members attached to the back region for fastening the article around the waist of the wearer.

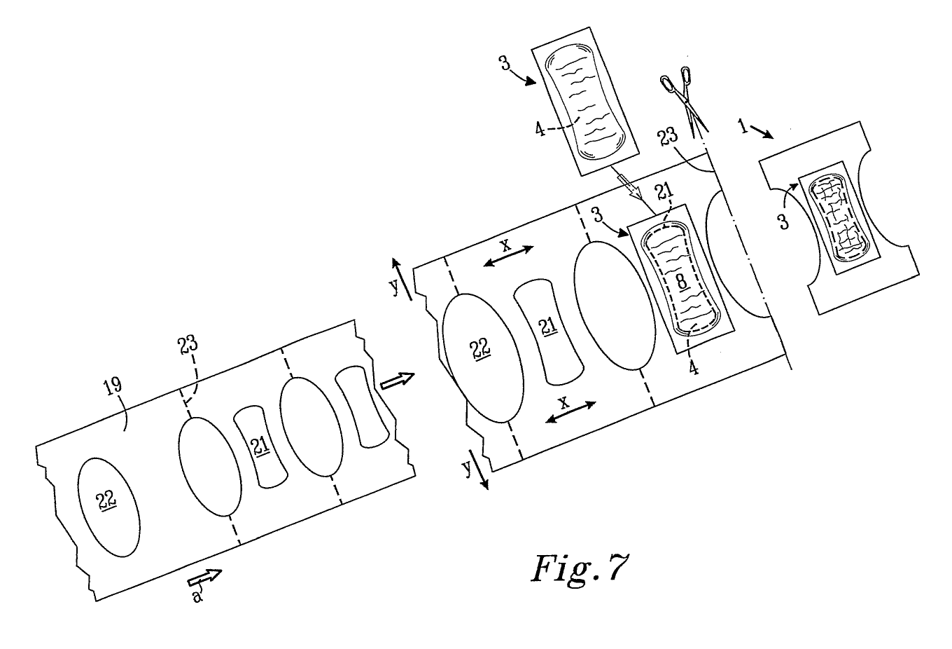

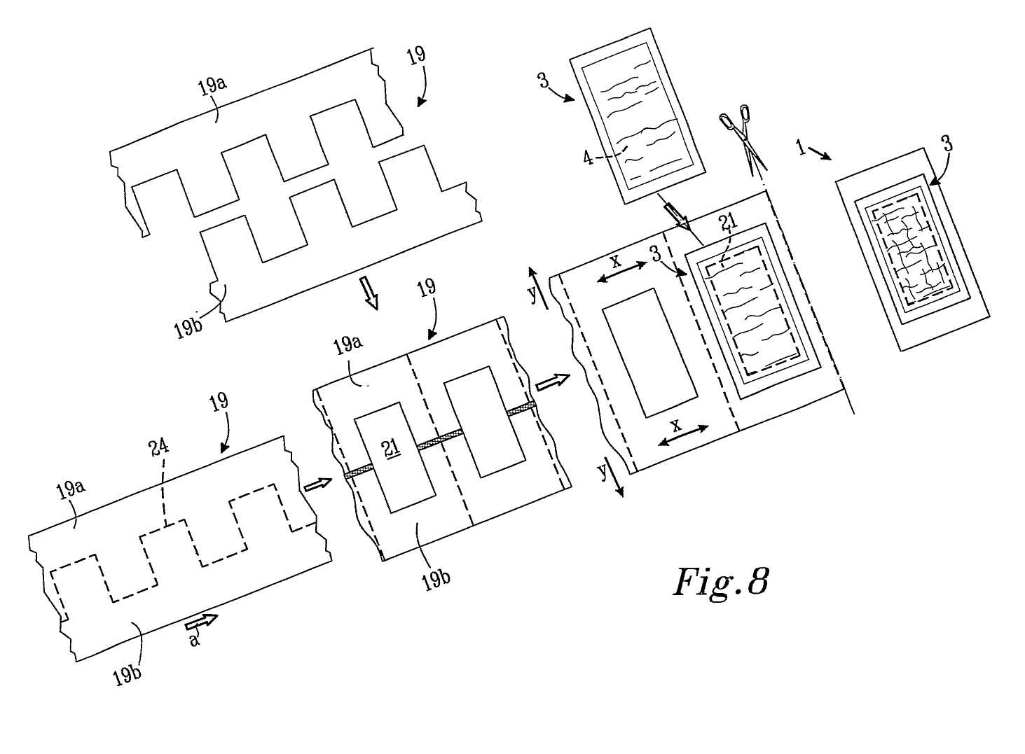

FIGS. 7 and 8 illustrate schematically two alternative methods for manufacturing an article according to the invention. According to FIG. 7 a web of elastic material 19 is advanced in a feeding direction a, said web having cutouts 22 made therein intended to form leg openings in an absorbent article. The intended lines of separation between individual articles are indicated with the numeral 23. Apertures 21 are made in the elastic web material 19 in the crotch region thereof. Said apertures 21 may be formed with the elastic web material 19 being in a non-extended or an extended state. In FIG. 7 it is shown that the apertures 21 are made while the elastic web material 19 is in a non-extended state.

The elastic web material 19 is subsequently extended in the longitudinal, y, and the transverse, x, direction of the article and an absorbent unit 3 comprising an absorbent core 4 is attached to the elastic web material 19 on the wearer facing side thereof so as to cover the aperture 21, while the elastic web material is in its extended state, in the manner described above. It is understood that the method steps of forming the aperture 21 in the elastic web material and extending said material may be reversed so that the aperture is made while the web is in an extended state. It may also be feasible to have the elastic web material stretched to a lower extent when forming the aperture therein and then stretching it further before attaching the absorbent unit 3 thereto. The elastic web material 19 is then cut into separate articles 1 along the lines 23 either before or after relaxation to the non-extended state.

FIG. 8 illustrates an alternative method for manufacturing an absorbent article in the form of an absorbent pad according to the invention, wherein it is avoided that elastic web material 19 is cut away in order to form the apertures 21. A staggered cutting line 24 is formed in the feeding direction a of the elastic web material, thus forming two separated webs 19a and 19b advanced in parallel in the feeding direction a. The web parts 19a and 19b are moved apart a first selected distance and one of the webs is displaced in the feeding direction a with respect to the other web a second selected distance, so that the two web parts 19a and 19b will form a configuration wherein apertures 21 are formed between them at regular intervals. Preferably there is a small overlap between the two web parts 19a and 19b, so that they may be joined to each other by suitable means, such as gluing, ultrasonic welding etc.

In a similar manner as described with reference to FIG. 7, the elastic web material 19 is subsequently extended in longitudinal, y, and transverse direction, x, and an absorbent unit 3 is attached to the elastic web material 19 so as to cover the aperture 21. The method steps of forming the staggered cutting line 21 and the apertures 21 in the elastic web material and extending said material may be reversed so that the staggered line and apertures are made while the web is in an extended state. The elastic web material 19 is then cut into separate articles 1.

It is understood that the methods for manufacturing the article may comprise additional steps such as adding waist elastics, fastening means, folding etc. The articles, both according to FIGS. 7 and 8, may also be manufactured in a longitudinal manufacturing line, thus advancing the articles in their longitudinal direction, y.

Although only a few embodiments of the present invention have been described and shown in detail, it is understood that many modifications are possible without departing from the scope of the present invention, which is defined in the claims.

1. An absorbent article comprising an elastic web material having an aperture formed therein, and an absorbent unit attached to a wearer facing side of the elastic web material covering said aperture and wherein the elastic web material surrounds the absorbent unit around all sides thereof, said article has a longitudinal direction and transverse direction, wherein said absorbent unit is attached to the elastic web material with the elastic web material being in an extended state of at least 30% in both the longitudinal and transverse directions of the article.

2. The absorbent article as claimed in claim 1, wherein the absorbent unit comprises an absorbent core and that said aperture in the elastic web material when this is in said extended state of at least 30%, when being attached to the absorbent unit, is smaller than but not more than 50% smaller than the surface area of the absorbent unit.

3. The absorbent article as claimed in claim 2, wherein the absorbent core has a shape similar to the shape of said aperture when the elastic web material is in said extended state of at least 30%, so that the contours of the aperture substantially follow the contours of the absorbent core.

4. The absorbent article as claimed in claim 2, wherein said aperture in the elastic web material when this is in said extended state of at least 30%, when being attached to the absorbent unit, is smaller than the absorbent core.

5. The absorbent article as claimed in claim 1, wherein the absorbent unit is attached to the elastic web material with the elastic web material being in an extended state of at least 40%, in at least one of the longitudinal and transverse directions of the article.

6. The absorbent article as claimed in claim 1, wherein the elastic web material has a higher extension in the longitudinal direction than in the transverse direction when being attached to the absorbent unit.

7. The absorbent article as claimed in claim 2, wherein the absorbent unit comprises an inner coversheet and an outer coversheet enclosing said absorbent core, said inner and outer coversheets extending beyond the peripheral edges of the absorbent core and being joined to each other.

8. The absorbent article as claimed in claim 7, wherein said outer coversheet has an external garment facing side comprising a fibrous web and that said elastic web material has a fibrous external garment facing surface.

9. The absorbent article as claimed in claim 1, wherein said article is a pant type absorbent article, said article comprising said absorbent unit, and a chassis surrounding the absorbent unit, said chassis comprising front, back and waist regions, while the absorbent unit is located at least in the crotch region of the article, said article in at least a part of the chassis comprises an outer coversheet in the form of said elastic web material having said aperture formed therein coinciding with the absorbent unit.

10. The absorbent article as claimed in claim 1, wherein the elastic web material extends beyond the peripheral edges of the absorbent unit around all sides thereof including in the crotch region of the article.

11. The absorbent article as claimed in claim 10, wherein said article is free from additional leg and/or crotch elastics.

12. A method for manufacturing an absorbent article comprising:
providing an elastic web material and forming an aperture therein;
stretching the elastic web material at least 30% in both longitudinal and transverse directions;
providing an absorbent unit and attaching said absorbent unit to the elastic web material while this is in its stretched state and so that the absorbent unit covers the aperture in the elastic web material.

13. The absorbent article as claimed in claim 5, wherein the absorbent unit is attached to the elastic web material with the elastic web material being in an extended state of at least 50%, in at least one of the longitudinal and transverse directions of the article.

14. The absorbent article as claimed in claim 9, wherein the article is a pant diaper, a sanitary pant or an incontinence pant.
